(19) United States
(12) Patent Application Publication
(10) Pub. No.: US 2023/0321250 A1
(43) Pub. Date: Oct. 12, 2023

(54) CANCER CELL MODULATORS

(71) Applicant: Octagon Therapeutics, Inc., Boston, MA (US)

(72) Inventors: Nathaniel Sherden, Boston, MA (US); Shen Yu, Winchester, MA (US)

(21) Appl. No.: 18/014,856

(22) PCT Filed: Jan. 29, 2021
(86) PCT No.: PCT/US2021/015845

§ 371 Date: Jan. 6, 2023

Related U.S. Application Data
(60) Provisional application No. 63/049,278, filed on Jul. 8, 2020.

(51) Int. Cl. A61K 47/54

(52) U.S. Cl. CPC A61K 47/543; A61K 47/542; A61K 47/549; A61K 47/548

(57) ABSTRACT

Disclosed are cancer cell- and proliferative cell-selective small molecule compounds that modulate certain cancer cell- or proliferative cell-specific metabolic enzymes or receptors, and methods of their use to treat cancer and other proliferative disorders.

### CROSS-REFERENCE TO RELATED APPLICATIONS

This application claims priority to U.S. provisional application No. 63/049,278 filed Jul. 8, 2020, entitled “Cancer Cell Modulators”, the contents of which are incorporated by reference herein in its entirety.

### FIELD OF THE INVENTION

The present invention is in the fields of medicine and proliferative diseases. More particularly, the invention relates to small molecule, cell-specific inhibitors and modulators of immune cell proteins for the treatment of cancer and other proliferative disorders.

### BACKGROUND OF THE INVENTION

Cancer and other proliferative disorders affect many throughout the world. Traditional treatments can control these disorders in some cases, but have not always cured them.

In addition to radiation therapy and surgical resection, other first generation therapies for cancer that demonstrated clinical utility were antiproliferative agents such as cancer chemotherapy agents. These compounds non-selectively affect any actively proliferating cells, usually by interfering in cellular metabolism, and thereby exhibit a range of toxicities including bone marrow suppression, GI-toxicity (by affecting intestine epithelial cells or crypt cells), and hair loss (by affecting hair follicles). Rapidly-proliferating lymphocytes are involved in normal immune function which is critical in controlling infection and in cancer surveillance. As a result, non-selective suppression of lymphocytes exposes patients to elevated risk of opportunistic infections as well as neoplasia.

In an effort to overcome these drawbacks, biologic therapies involving the use of antibodies specific for certain targets on cancer cells and that can modulate specific inflammatory or effector pathways have been developed. Antibodies target molecules are only expressed on the surface of the cancer cells (such as CTLA4 and CD20) as antibodies are larger molecular weight molecules that do not penetrate the cell

Unfortunately, many of these immunotherapies may have other significant drawbacks. For example, the anti-CD20 antibody. Rituximab (Rituxan), widely used in B-cell malignancy treatment, triggers cell death via antibody-dependent cellular cytotoxicity (ADCC) when it binds to CD20 on a B-cell surface. However, Rituximab is also known to cause headache and back pain, in addition to possessing a slow administration infusion rate (50 mg/hr), which can take up to eight hours. In addition, its administration increases the risk of infections and malignancies as it depletes B-cells; normal B-cell functions are essentially absent for patients treated with Rituximab. Treatments like Intravenous Immune Globulin (IVIG) can partially restore B-cell functions, but this method also has severe toxicity liabilities.

In contrast, small molecule cancer drugs are much smaller in size (≤500 Da) and thus can translocate through plasma membranes and often are amenable to oral administration. Thus, small molecule cancer drugs have been successfully used to target extracellular, cell surface ligand-binding receptors as well as the intracellular proteins, including metabolic enzymes and anti-apoptotic proteins that play a key role in transducing downstream signaling for cell growth and metastasis promotion. Representative inhibitors include kinase inhibitors, thymidylate synthase inhibitors and dihydrofolate reductase (DHFR) inhibitors. However, the activity of these small molecule therapies also can affect any cells they encounter, including noncancerous cells, which have that target.

Thus, improved small molecule antiproliferative drugs that are cancer cell type-selective and disease-modifying are highly desired.

### SUMMARY OF THE INVENTION

It has been discovered that small molecule chemotherapeutic drugs that are modulators of certain metabolic enzymes found in cancer cells and modulators of certain receptors found on cancer cells can be chemically optimized for guidance to, and selection for, targeted proliferating cell types, and that these compounds exhibit a high level of anti-proliferative activity in cell-based assays.

These discoveries have been exploited to develop the present disclosure, which, in part, is directed to cancer- andr proliferative cell-selective inhibitor or modulator compounds (“CPSIs” or “CPSI compounds”), and to methods of their synthesis and their use to treat proliferative cell disorders.

In one aspect, the disclosure provides a cancer- or proliferative cell-selective inhibitor or modulator compound (“CPSI”) comprising: a bait comprising a molecule which targets a cancer cell or a cell affected by a proliferative disorder, the bait comprising a substrate cleavable by a disease factor present in the cancer cell or the cell affected by a proliferative disorder; and a payload comprising an active inhibitor or modulator of a metabolic enzyme found in the cancer cell or in the cell affected by a proliferation disorder, the payload comprising an antiproliferative agent.

In one embodiments, in the CPSI compound, the bait is selected from an oligomer comprising 1-5 units, which are, independently at each occurrence selected from the group consisting of

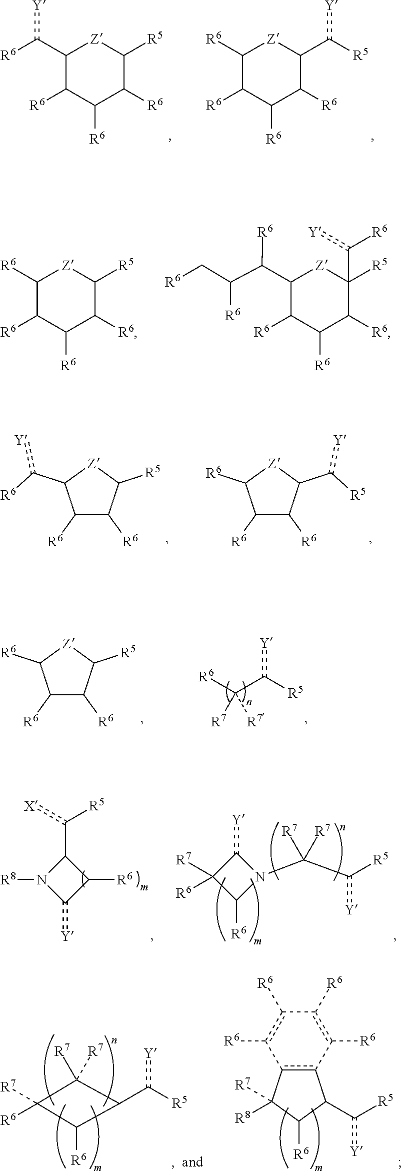

wherein:
Z′ is independently, at each occurrence, selected from the group consisting of CH₂, NH, O, and S;
X′ and Y′ are independently, at each occurrence, selected from the group consisting of O, NH, and S;
R⁵ is independently, at each occurrence, selected from the group consisting of a bond to any R⁶, a bond to any R⁸, and an attachment to any payload
R⁶ is independently, at each occurrence, selected from the group consisting of H, —R^{D}, —R^{A}—R⁵, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H. C₁₋₄ alkyl, C₂₋₄ alkenyl, C₂₋₄ vinyl, and C₃₋₄ allyl, all of which are optionally substituted with one, two, three, or four halo;
R^{D} is independently, at each occurrence, selected from the group consisting of OH SH, NH₂, N₃, and halo;
R⁷ and R⁷′ are independently, at each occurrence, selected from the group consisting of H, NH₂, OH, C₆₋₁₀ aryl, 5-10 membered heteroaryl, C₁₋₄ alkyl, —(CH₂)₁₋₃—C₆₋₁₀ aryl, and —(CH₂)₁₋₃-5-10 membered heteroaryl; wherein aryl, heteroaryl, and alkyl are optionally substituted with one, two, or three substituents selected from the group consisting of halo, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, —R^{A}—(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵;
   m is 0, 1, 2, 3, or 4; and
   n is 0, 1, 2, or 3.

In another embodiment, the Bait is selected from the group consisting of

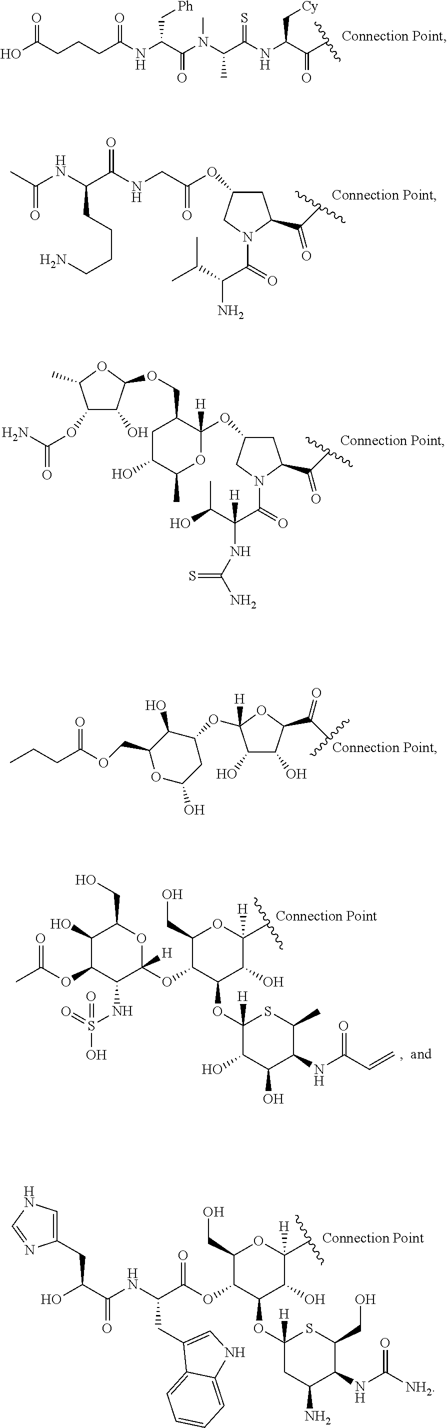

In some embodiments, in the CPSI compound, the payload modulates or inhibits an enzyme selected from the group consisting of thymidylate synthase, proteasome, dihydrofolate reductase, ribonucleotide reductase, DNA methyltransferase, glycinamide ribonucleotide formyltransferase, adenosine deaminase, glutamine-phosphoribosyl pyrophosphate, amidotransferase, tyrosine-protein kinase, spleen tyrosine kinase, phosphatidylinositol 3-kinases, phosphoinositide 3-kinases. Bruton's tyrosine kinase, histone deacetylases, and Janus kinase (JAK), XPO1, and bromodomain and extra-terminal domain (BET) proteins.

In other embodiments, the payload modulates or inhibits a receptor selected from the group consisting of epidermal growth factor receptor, mammalian target of rapamycin (mTOR), and BCL2.

In certain embodiments, the payload comprises Bortezomib or a derivative thereof. In a specific embodiment, the CPSI compound comprises:

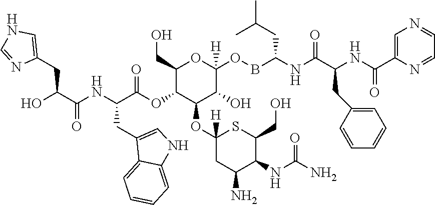

or a pharmaceutically acceptable salt thereof.

In another embodiment, the payload comprises Lestaurtinib or a derivative thereof. In a specific embodiment, the CPSI comprises:

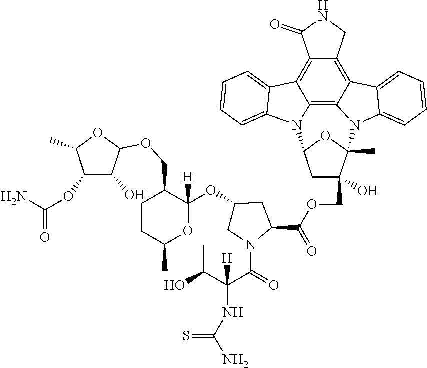 [SMILES: C[C@H](O)[C@@H](NC(N)=S)C(=O)N1C[C@H](O[C@@H]2O[C@@H](C)CC[C@H]2COC2O[C@@H](C)[C@@H](OC(N)=O)[C@H]2O)C[C@H]1C(=O)OC[C@@]1(O)C[C@H]2O[C@]1(C)n1c3ccccc3c3c4c(c5c6ccccc6n2c5c31)C(=O)NC4]

or a pharmaceutically acceptable salt thereof.

In another embodiment, the payload comprises Umbralisib or a derivative thereof. In a specific embodiment, the CPSI compound comprises:

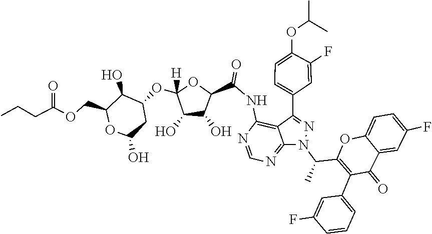

or a pharmaceutically acceptable salt thereof.

In yet another embodiment, in the CPSI compound, the payload comprises Floxuridine or a derivative thereof. In a specific embodiment, the CPSI compound comprises:

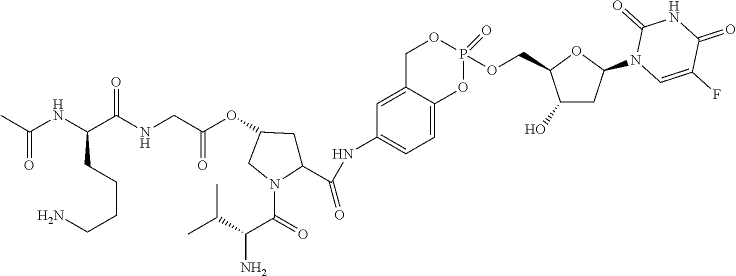

or a pharmaceutically acceptable salt thereof.

In another embodiment, in the CPSI compound, the payload comprises 6-thioguanine or a derivative thereof. In a specific embodiment, the CPSI compound comprises:

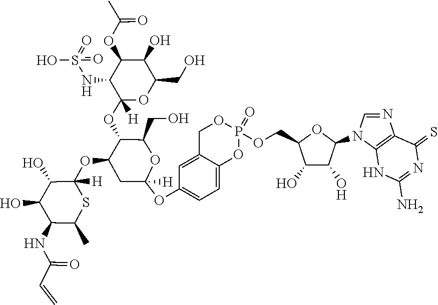 [SMILES: C=CC(=O)N[C@H]1[C@@H](O)[C@H](O)C[C@H](O[C@@H]2C[C@H](Oc3ccc4c(c3)COP(=O)(OC[C@H]3O[C@@H](n5cnc6c(=S)nc(N)[nH]c65)[C@H](O)[C@@H]3O)O4)O[C@H](CO)[C@H]2O[C@H]2O[C@H](CO)[C@H](O)[C@H](OC(C)=O)[C@H]2NS(=O)(=O)O)S[C@H]1C]

or a pharmaceutically acceptable salt thereof.

In some embodiments, the CPSI compound further comprising a linker molecule attached to the bait and attaching the payload to the bait. In certain embodiments, the linker is selected from the group consisting of

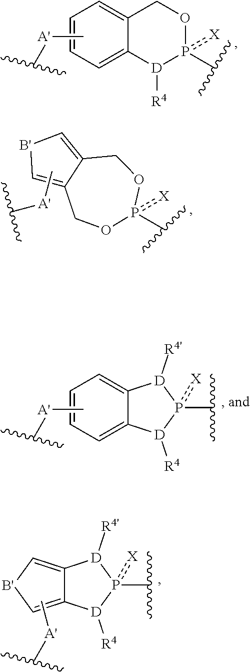

wherein:
A′ is independently, at each occurrence, selected from the group consisting of CH₂, NH, O, S, CO₃, CO₂N, CO₂S, and CO₂N;
B′ is independently, at each occurrence, selected from the group consisting of CH₂, NH, O, and S;
D and D′ are each independently selected from the group consisting of O, N, NH, and S;
X is independently selected at each occurrence from the group consisting of O, NH, and S; and
R⁴ and R⁴′ are each independently selected from the group consisting of H, C₁₋₄ alkyl, C₂₋₄ alkenyl, C₂₋₄ vinyl, and C₃₋₄ allyl, all of which are optionally substituted with one, two, three, or four halo.

In another aspect, the disclosure provides a pharmaceutical formulation comprising a CPSI compound as described above, or a pharmaceutically acceptable salt thereof, and a pharmaceutically acceptable carrier. In some embodiments, the pharmaceutical formulation further comprises an anti-proliferative compound that is not the CPSI compound.

In still another aspect, the disclosure provides a method of treating a cancer or proliferative disorder in a subject, comprising administering to the patient a pharmaceutical formulation comprising a CPSI as described herein, in an amount effective to reduce or inhibit at least one symptom of the cancer or proliferative disorder.

In some embodiments, the pharmaceutical formulation administered comprises a CPSI compound of Formula I, II, III, IV, or a pharmaceutically acceptable salt thereof. In other embodiments, the pharmaceutical formulation comprises a CPSI compound of Formula VI, VII, VIII, or a pharmaceutically acceptable salt thereof.

In certain embodiments, the proliferative disorder is a solid cancer or a liquid cancer. In specific embodiments, the liquid cancer is Acute Lymphocytic Leukemia, T-Acute Lymphocytic Leukemia, Peripheral T Cell Lymphoma, Acute Myeloid Leukemia, Myelodysplastic Syndromes, Hairy Cell Leukemia, Mantel Cell Lymphoma, Chronic Lymphocytic Leukemia, B-Chronic Lymphocytic Leukemia, Chronic Myeloid Leukemia. Non-Hodgkin's Lymphoma, Myeloproliferative Neoplasms, or Polycythemia vera. In other embodiments, the solid cancer is gastric cancer, Non-Small Cell Lung Cancer, Pleural Mesothelioma, breast cancer, colon cancer, pancreatic cancer, bladder cancer, cervical cancer, Osteosarcoma, Head and Neck cancer, prostate cancer, testicular cancer, or ovarian cancer

In some embodiments, the method further comprising administering to the patient a therapeutically effective amount of an anti-proliferative agent that is not a CPSI.

The disclosure also provides a method of synthesizing a CPSI according to the disclosure, comprising carrying out the method set forth in FIGS. 1-3.

In yet another aspect, the disclosure provides a method of modulating the activity of a cancer or proliferative cell, comprising contacting the cell with a CPSI compound according to the disclosure in an amount effective to reduce or inhibit the proliferative activity of the cell.

### DESCRIPTION OF THE DRAWING

The foregoing and other objects of the present disclosure, the various features thereof, as well as the disclosure itself may be more fully understood from the following description, when read together with the accompanying drawings in which:

FIG. 1 is a diagrammatic representation of the acquisition of a representative Payload, wherein all boxed entities represent chemical mater, either a whole molecule, or where joined by a line, represent a portion of a molecule connected by one or more chemical bonds to the other portion(s) as indicated by the connecting line; arrows indicate actions such as synthetic step(s); solid lines for both boxes and arrows represent required steps and molecules, or portions thereof; and dashed lines for boxes and arrows represent steps and molecules or portions thereof that may be conditional:

FIG. 2 is a diagrammatic representation of a synthesis of a representative Bait, wherein: all boxed entities represent chemical mater—either a whole molecule or where joined by a line represent a portion of a molecule connected by one or more chemical bonds to the other portion(s) as indicated by the connecting line; arrows indicate actions such as synthetic step(s), solid lines for both boxes and arrows represent required steps and molecules or portions thereof; and dashed lines for boxes and arrows represent steps and molecules or portions thereof that may be conditional;

FIG. 3 is a diagrammatic representation of the final assembly of a representative CSIM according to the disclosure, wherein all boxed entities represent chemical mater—either a whole molecule or where joined by a line represent a portion of a molecule connected by one or more chemical bonds to the other portion(s) as indicated by the connecting line; arrows indicate actions such as synthetic step(s), solid lines for both boxes and arrows represent required steps and molecules or portions thereof, and dashed lines for boxes and arrows represent steps and molecules or portions thereof that may be conditional.

### DESCRIPTION

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The terms “affected cell” and “affected proliferating cell” encompasses a cell which is proliferating abnormally or is proliferating in response to an abnormal signal, such as, but not limited to, cancer, tumor, autoimmune, and dermatologic disorders such psoriasis, eczema, and ichthyosis and other abnormally proliferating cells such as autoimmune disorders

The term “treat,” “treated,” “treating,” or “treatment” includes the diminishment or alleviation of at least one symptom associated or caused by the state, disorder or disease being treated. In certain embodiments, the treatment comprises bringing into contact with an infection an effective amount of an anti-infective formulation of the disclosure for conditions related to infections.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, but are not limited to, livestock and pets, such as ovine, bovine, porcine, canine, feline and marine mammals.

As used herein, the term “modulates” or “modulator” refers to the ability of a CPSI to change (e.g., to inhibit, to stop, to reduce, to block, or to increase)) the activity of a protein such as an enzyme in, or receptor on, the immune cell to which it is directed or targeted, resulting in a change in the physiology of the immune cell, including but not limited to, their proliferation rates, production of cytokines (e.g., interleukins, TNFs, interferons), chemokines, immunoglobulins, or other secreted factors, or immune response towards pathogens such as phagocytosis. The changes may also be the response of the targeted immune cells to stimulus, for example, by mitogens, antigens, secreted factors from other immune and non-immune cells, or direct interaction with other immune and non-immune cells.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “proliferation disorder” refers to excessive, abnormal proliferation or growth of cells and turnover of cellular matrix contribute significantly to the pathogenesis of diseases, including, but not limited to, solid and liquid cancers, tumors, inflammatory diseases such as atherosclerosis (AS), and many autoimmune diseases such as rheumatoid arthritis (RA), inflammatory bowel diseases (IBD), psoriasis as well as infectious diseases of bacterial, viral or fungal nature. A “proliferative” cell is a cell undergoing excessive, abnormal growth.

As used herein, the term “pharmaceutically acceptable” refers to a material, such as a carrier or diluent, which does not abrogate the biological activity or properties of the CPSI compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Nonlimiting examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids.

As used herein, the term “composition” refers to the CPSI.

The term “pharmaceutical composition”, or “formulation” refers to at least one of the same or different CPSI or/and salt thereof, according to the disclosure in a pharmaceutically acceptable carrier, and may encompasses other components such as at least one immunomodulatory or antiproliferative compound that is not a CPSI according to the disclosure

An “oral dosage form” includes a unit dosage form prescribed or intended for oral administration.

As used herein, the term “proliferation disorder” refers to excessive, abnormal proliferation or growth of cells and turnover of cellular matrix contribute significantly to the pathogenesis of diseases, including, but not limited to, cancer, tumor, atherosclerosis, rheumatoid arthritis, psoriasis, idiopathic pulmonary fibrosis, scleroderma and cirrhosis of the liver.

As used herein, the term “alkyl.” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. In an embodiment, C₁-C₆ alkyl groups are provided herein. Nonlimiting examples include methyl, ethyl, propyl, isopropyl, butyl, isobutyl, tert butyl, pentyl, neopentyl, and hexyl. Other nonlimiting examples of C₁-C₆ alkyl include ethyl, methyl, isopropyl, isobutyl, n-pentyl, and n-hexyl.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. For example, the alkenyl moiety contains 2 to 6, 2 to 4, or 2 to 3 carbon atoms. Exemplary alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “allyl” employed alone or in combination with other terms, refers to an sp³ carbon atom adjacent to an alkenyl group as defined above.

As used herein, the term “halo” or “halogen” alone or as part of another substituent means, unless otherwise stated, a fluorine, chlorine, bromine, or iodine atom.

As used herein, the term “aromatic” refers to a carbocycle or heterocycle with one or more polyunsaturated rings and having aromatic character, i.e., having (4n+2) delocalized a elections where n is an integer).

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. Aryl groups may have 6 carbon atoms, six to ten carbon atoms, or six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thiophenyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]-triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl.

It is to be understood that if an aryl and heteroaryl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, and the term “thienyl” means 2- or 3-thioenyl.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The present disclosure provides small molecule cancer- or proliferative cell-selective inhibitor or proliferative cell-selective modulator compounds (“CPSIs” or “CPSI compounds”) that are proliferative disease-modifying and immunomodulatory. These CPSIs target and inhibit or modulate certain proteins such as enzymes found in rapidly proliferating cells or cancer cells, or which modulate receptors found on cancer or rapidly proliferating cells.

### CPSI Targeted Cell Types

CPSIs of the disclosure are targeted to both solid tumors and liquid cancers or hematological malignancies, as well as to other cells affected by a proliferation disorder. Exemplary cancer cell types to which CPSIs of the disclosure are targeted include, but are not limited to, those in Table 1 below.

TABLE 1

| | | |
|---|---|---|
| CANCER CELL | | |
| (Abbrev.) | CANCER | CELL TYPES |

Hematological Cancers

| | | |
|---|---|---|
| ALL | Acute Lymphocytic Leukemia | Lymphocytes (B, T, NK cells) |
| T-ALL | T-Acute Lymphocytic | T Cells |
| | Leukemia | |
| PTCL | Peripheral T Cell Lymphoma | T Cells |
| AML | Acute Myeloid Leukemia | Cells of myeloid origin including |
| | | myeloid stem cells, myeloid blast, |
| | | neutrophils |
| MDS | Myelodysplastic Syndromes | Cells of myeloid origin including |
| | | myeloid stem cells, myeloid blast, |
| | | neutrophils |
| HCL | Hairy Cell Leukemia | B Cells |
| MCL | Mantle Cell Lymphoma | B Cells |
| CLL | Chronic Lymphocytic | Lymphocytes (B, T, NK cells) |
| | Leukemia | |
| B-CLL | B-Chronic Lymphocytic | B Cells |
| | Leukemia | |
| CML | Chronic Myeloid Leukemia | Cells of myeloid origin including |
| | | myeloid stem cells, myeloid blast, |
| | | neutrophils |
| NHL | Non-Hodgkin's Lymphoma | Lymphocytes (B, T, NK cells) |
| MPN | Myeloproliferative | Cells of hematopoietic origin |
| | Neoplasms | |
| PCV | Polycythemia vera | Cells of hematopoietic origin |

Solid Tumors

| | | |
|---|---|---|
| GC | Gastric Cancer | Epithelial cells at glandular |
| | | epithelium of the gastric mucosa; |
| | | Gastric signet ring cells; or |
| | | Gastric B-cells |
| NSCLC | Non-Small Cell Lung Cancer | Lung epithelial cells |
| PM | Pleural Mesothelioma | Mesothelial cells that lines lungs, |
| | | stomach, heart and other organs |
| BC | Breast Cancer | Milk duck cells; Lobule cells |
| CC | Colon Cancer | Epithelial cells that line the colon |
| | | or rectum |
| PC | Pancreas Cancer | Pancreatic epithelial cells |
| BLC | Bladder Cancer | Transitional (urothelial) cells, |
| | | squamous cells or epithelial cells |
| | | in the bladder |
| HNC | Head and Neck Cancer | Squamous cells or epithelial cells |
| | | in mouth, nose, throat, |
| | | nasopharynx, hypopharynx, |
| | | larynx and trachea; cells in the |
| | | salivary gland |
| CVC | Cervical cancer | Squamous and epithelial cells at |
| | | the cervix |
| OS | Osteosarcoma | Primitive transformed cells of |
| | | mesenchymal origin surrounding |
| | | bones |

Targeting of CPSIs to a particular cancer cell or to an abnormally proliferating cell type is accomplished via a moiety that serves as a substrate for one or more enzymes present only in the targeted cell, or which specifically binds to a receptor present on the targeted cells. Cell type specificity can be conferred in a variety of ways. Differential permeability, differential activation of a prodrug, differential degradation, and differential enzymatic activation, and/or differential expulsion from different cell types are nonlimiting representative ways in which a drug can be activated preferentially in one cell type compared to others. For example, CPSIs according to the disclosure may be optimized to achieve cancer cell-selective accumulation based on interaction with a receptor or enzymes that are highly expressed on or in in the particular cancer cell.

### CPSIs

The CPSIs of the present disclosure achieve their uniqueness from their structure, which may consist of three parts: (1) a “bait” or selective moiety, which is a substrate that can be cleaved e.g., by an enzyme that is highly expressed in the target cell type; (2) a “payload” moiety, which is the active inhibitor or modulator of a metabolic enzyme or receptor found in or on selected cancer cells and is an antiproliferative agent; and (3) a “linker” moiety, which is unstable on its own but can be stabilized by being bound to the bait and is placed between the bait and the payload.

### BAIT—LINKER—PAYLOAD

The CPSIs can have one or more linkers attached to one or more baits and/or payloads. Some nonlimiting, representative linker moieties comprise:

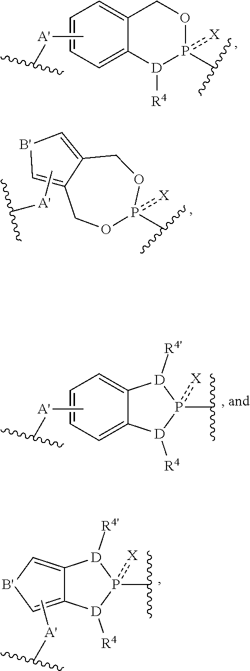

wherein:
A′ and B′ are each independently selected from the group consisting of CH₂, NH, O, and S;
D and D′ are each independently selected from the group consisting of O, N, NH, and S;
X is selected from the group consisting of O, NH, and S; and
R⁴ and R⁴′ are each independently selected from the group consisting of H, C₁₋₄ alkyl, C₂₋₄ alkenyl, C₂₋₄ vinyl, and C₃₋₄ allyl, all of which are optionally substituted with one, two, three, or four halo.

Other CPSIs of the present disclosure do not have a linker component. Instead, the bait is directly linked to the payload.

### BAIT—PAYLOAD

The payload moiety comprises a fully active inhibitor or/modulator of a metabolic enzyme in, or receptor on, a cancer or affected cell and which does not require a conversion step to become therapeutic. Thus, CPSIs comprise active payloads, including the active species of a normally inactive payload compound that naturally gets metabolically converted to the active species within the body.

The bait is an oligomer comprising about 1-5 monomer units which can be one or multiples of the same or a combination of different small organic molecules such as, but not limited to, amino acids, sugars, and/or lipids, including, but not limited to, those existing in nature. This component optimizes the CPSIs to achieve cancer- or abnormally proliferating-cell-selective accumulation by serving, for example, as a substrate for one or more enzymes present in the targeted cells or which binds to a receptor found on the targeted cells. It also serves to stabilize the CPSI compound, assisting in inhibiting the activity of the payload by restricting cell permeability or by electrostatic or steric interference with regions on the target, for example. Thus, the activity of the payload in the CPSI is inhibited or inactive until the bait component is released.

Cleavage of the bait or selectivity moiety and release of the payload occurs when disease factors are present, thus sparing unaffected cells. As used herein, the term “disease factors” encompasses compounds and molecules present in the environment of the cancer or affected cells and which are associated with the cancer or affected cells, and may be synthesized or elicited by the cancer or affected cells, or the presence thereof. Representative, nonlimiting disease factors include small molecules (chemical compounds) or macromolecules that circulate in plasma of a subject with active cancer-related diseases, but not in healthy subjects or subjects that have their diseases in remission. The presence of a disease factor results in the production or activation of the enzyme or receptor that causes or results in the cleavage of the bait from the CPSI.

As used herein, the term “bait” is an oligomer comprising 1-5 units, which can be independently, at each occurrence, selected from the group consisting of

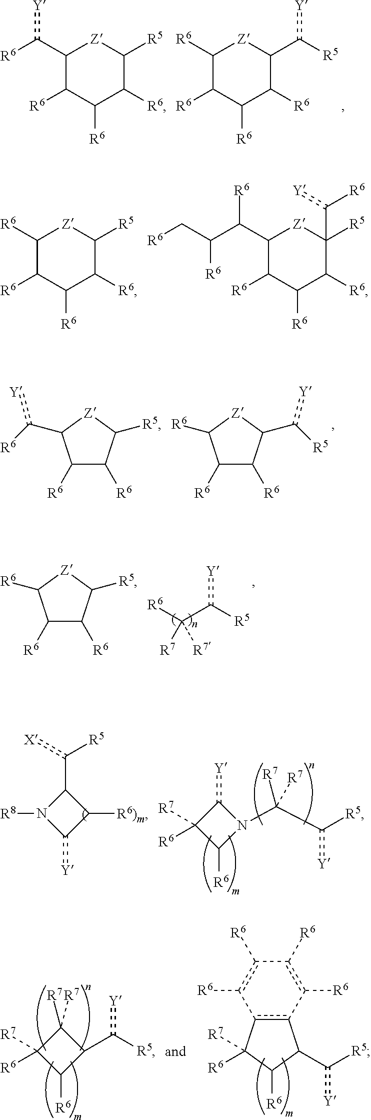

wherein:
Z′ is independently, at each occurrence, selected from the group consisting of CH₂, NH, O, and S;
X′ and Y′ are independently, at each occurrence, selected from the group consisting of O, NH, and S;
R⁵ is independently, at each occurrence, selected from the group consisting of a bond to any R⁶, a bond to any R⁸, and an attachment to any of the payloads listed below via the point in the depicted structure shown as attached to the generic “bait.
R⁶ is independently, at each occurrence, selected from the group consisting of H, —R^{D}, —R^{A}—R⁵, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{D} is independently, at each occurrence, selected from the group consisting of OH, SH, NH₂, N₃, and halo;
R⁷ and R⁷′ are independently, at each occurrence, selected from the group consisting of H, NH₂, OH, C₆₋₁₀ aryl, 5-10 membered heteroaryl. C₁₋₄ alkyl, —(CH₂)₁₋₃—C₆₋₁₀ aryl, and —(CH₂)₁₋₃-5-10 membered heteroaryl; wherein aryl, heteroaryl, and alkyl are optionally substituted with one, two, or three substituents selected from the group consisting of halo, ═O, ═NH, ═S, —R^{A}—(C═R^{B})—R^{C}, —R^{A}—(C═R^{B})—R^{A}—R^{C}, R^{A}—(SO₂)—R^{D}, —R^{A}(SO₂)—R^{C}, —R^{A}—(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵;
m is 0, 1, 2, 3, or 4; and
n is 0, 1, 2, or 3.

Representative, nonlimiting baits are

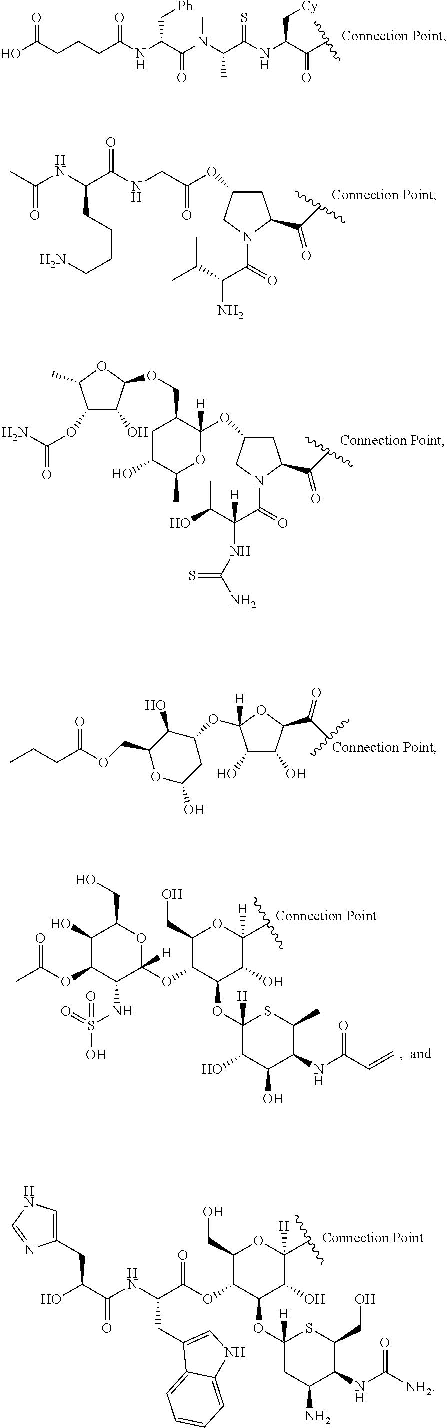

Nonlimiting representative metabolic enzymes that are active in some representative cancer and other proliferating cells, as well as nonlimiting, representative receptors found on such cells, and their respective payloads that can inhibit or modulate them, include, but are not limited to, those in Table 3 below.

TABLE 3

| | |
|---|---|
| Target | Exemplary Payloads |

Targets for Chemotherapy

| | |
|---|---|
| Thymidylate synthase | TS-1 (Tegafur/gimeracil/oteracil) |
| | (Teysuno); Pemetrexed (Alimta); |
| | Capecitabine (Xeloda); 5-Fluoro-2′- |
| | deoxyuridine (floxuridine, FUDR); 5- |
| | Fluorouracil (Efudex, Adrucil) |
| Dihydrofolate reductase (DHFR) | Pralatrexate (Folotyn); Pemetrexed |
| | (Alimta); Methotrexate (amethopterin, |
| | Trexall, Rheumatrex) |
| Ribonucleotide reductase | Clofarabine (Clolar); Arabinofuranosyl-2- |
| | fluoroadenine (fludarabine; Fludara); |
| | Hydroxyurea (Hydrea) |
| DNA methyltransferase | 5-Aza-2′-deoxycytidine (decitabine; |
| | Dacogen); Azacitidine (Vidaza) |
| Glycinamide Ribonucleotide Formyltransferase | Pemetrexed (Alimta) |
| (GARFT) | |
| Adenosine deaminase | 2′-Deoxycoformycin (pentostatin, Nipent) |
| Glutamine-phosphoribosyl pyrophosphate | 6-Thioguanine (Lanvis); 6- |
| amidotransferase (GPAT) | Mercaptopurine (Purinethol) |
| GTP-binding protein Rac1 | 6-Thioguanine (Lanvis); 6- |
| | Mercaptopurine (Purinethol) |
| Purine Synthesis Enzymes | O⁶-Methylarabinofuranosyl guanine |
| | (nelarabine; Arranon) |
| DNA/RNA incorporation | 5-Aza-2′-deoxycytidine (decitabine; |
| | Dacogen); Azacitidine (Vidaza); 2′,2′- |
| | Difluoro-2′-deoxycytidine (gemcitabine; |
| | Gemzar); 2-Chloro-2′-deoxyadenosine |
| | (cladribine; Leustatin); 5-Fluoro-2′- |
| | deoxyuridine (floxuridine, FUDR); |
| | Arabinofuranosylcytosine (cytarabine; |
| | Cytosar-U); 6-Thioguanine (Lanvis); 6- |
| | Mercaptopurine (Purinethol) |

Targets for Targeted Cancer Therapies

| | |
|---|---|
| Epidermal Growth Factor Receptor (EGFR) | Gefitinib; Erlotinib; Afatinib; Brigatinib; |
| | Icotinib: Osimertinib |
| Mammalian Target of Rapamycin (mTOR) | Temsirolimus; Sirolimus; Everolimus |
| BCL2 | Venetoclax; Navitoclax; ABT-737 |
| Tyrosine-protein kinase (or Spleen Tyrosine | Fostamatinib; TAK-659; Cerdulatinib; |
| Kinase, Syk) | Entospletinib; Nilvadipine |
| Phosphatidylinositol 3-Kinases (or | Wortmannin; LY294002; Duvelisib; |
| Phosphoinositide 3-kinases, PI3K) | Idelalisib; Copanlisib; Buparlisib; |
| | Umbralisib |
| Bruton's tyrosine kinase (BTK) | Ibrutinib; Acalabrutinib; Zanubrutinib; |
| | Evobrutinib; GS-4059 (ONO-4059); |
| | ABBV-105; Spebrutinib (AVL-292, CC- |
| | 292); HM71224 |
| Histone Deacetylases (HDAC) | Vorinostat; Mocetinostat; CUDC-907; |
| | Valproic Acid; Istodax (romidepsin); |
| | Trichostatin A (TSA); Belinostat |
| | (PXD101); LAQ824; Panobinostat |
| | (LBH589); Entinostat (MS-275); |
| | Tacedinaline (CI994) |
| Janus Kinase (JAK) | Ruxolitinib (Jakafi, Jakavi); Cerdulatinib |
| | (PRT062070); Gandotinib (LY-2784544); |
| | Lestaurtinib (CEP-701); Momelotinib |
| | (GS-0387, CYT-387); Pacritinib |
| | (SB1518); CHZ868; Cucurbitacin I (JSI- |
| | 124) |
| Proteasome | Bortezomib; Ixazomib |
| XPO1 | Selinexor |
| BET | OTX015; CPI-0610 |

Representative CPSI compounds according to the disclosure can comprise: all amino acid and simple substituents (amide caps, etc. . . . ) in a linear fashion; all amino acid and simple substituents in a branched fashion; amino acid with some sugar substituents in a branched fashion.

Representative CPSI compounds that act as modulators or inhibitors of cancer cell or proliferative cell enzymes can fall into the generic structures of formulae I-IV set forth below.

Representative, nonlimiting CPSI compounds fall into the generic structure of Formula I:

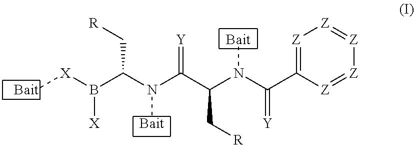

or a pharmaceutically acceptable salt thereof, wherein:
---- is an optional bond;
R is selected from the group consisting of H, C₆₋₁₂ aryl, C₁₋₆ alkyl, C₂₋₆ alkenyl, C₂₋₆ vinyl, and C₃₋₆ allyl;
X is selected from the group consisting of O, S, and NH;
Y is selected from the group consisting of O and S;
Z is selected from the group consisting of N and CH; and
the compound contains at least one of the optional bonds to “Bait”.

A nonlimiting, representative CPSI compound of Formula 1 including the payload Bortezomib comprises:

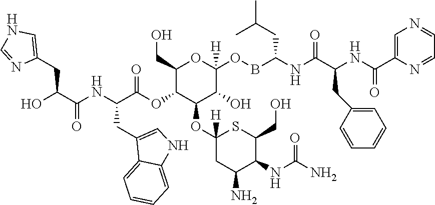

or a pharmaceutically acceptable salt thereof.

Representative, nonlimiting CPSI compounds fall into the generic structure of Formula II:

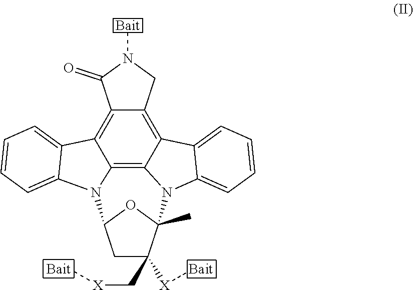

or a pharmaceutically acceptable salt thereof, wherein:
---- is an optional bond;
X is selected from the group consisting of O, S, NH; and
the compound contains one or more the optional linkages to “Bait”.

A nonlimiting, representative CPSI compound of Formula II including the payload Lestaurtinib comprises:

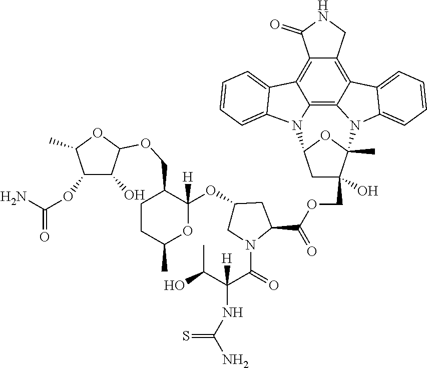 [SMILES: C[C@H](O)[C@@H](NC(N)=S)C(=O)N1C[C@H](O[C@@H]2O[C@@H](C)CC[C@H]2COC2O[C@@H](C)[C@@H](OC(N)=O)[C@H]2O)C[C@H]1C(=O)OC[C@@]1(O)C[C@H]2O[C@]1(C)n1c3ccccc3c3c4c(c5c6ccccc6n2c5c31)C(=O)NC4]

or a pharmaceutically acceptable salt thereof.

Representative, nonlimiting CPSI compounds fall into the generic structure of Formula III:

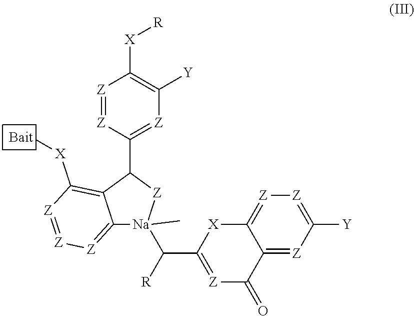

or a pharmaceutically acceptable salt thereof, wherein:
R is selected from the group consisting of H, C₆₋₁₂ aryl, C₁₋₆ alkyl, C₂₋₆ alkenyl, C₂₋₆ vinyl, and C₃₋₆ allyl;
X is selected from the group consisting of O, S, and NH;
Y is selected from the group consisting of H, halogen, OH, and NH₂; and
Z is selected from the group consisting of N and CH.

A nonlimiting, representative CPSI compound of Formula III including the payload Umbralisib comprises:

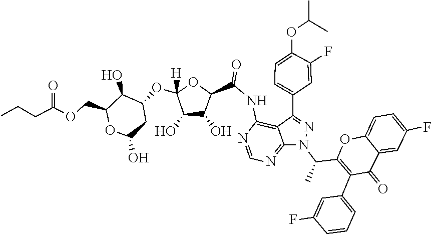

or a pharmaceutically acceptable salt thereof.

Representative, nonlimiting CPSI compounds fall into the generic structure of Formula IV:

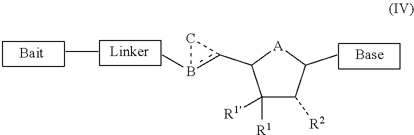

or a pharmaceutically acceptable salt thereof, wherein:
---- is an optional bond;
A is selected from the group consisting of CH₂, NH, O, and S;
B is selected from the group consisting of C, CH, CH₂, N, NH, O, and S;
C is selected from the group consisting of CH₂, NH, O, and S;
R¹ is selected from the group consisting of H, C₁₋₄ alkyl, C₂₋₄ alkenyl, C₂₋₄ vinyl, and C₃₋₄ allyl, all of which are optionally substituted with one, two, three, or four halo;
R¹′ is selected from the group consisting of OH SH, NH₂, N₃, and halo;
R² is selected from the group consisting of OH, SH, NH₂, N₃, and halo;
Base is selected from the group consisting of

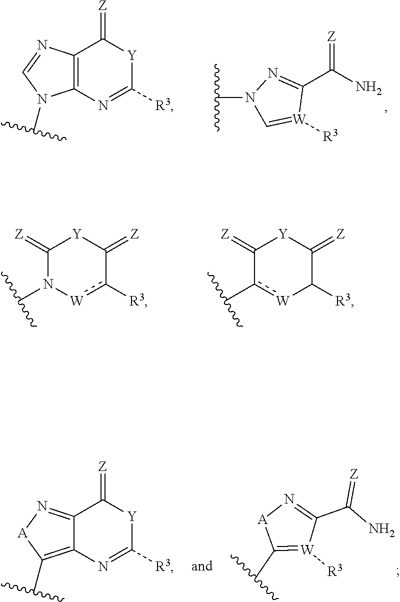

Z is selected from the group consisting of O, NH, and S;
Y is selected from the group consisting of O, NH, and S;
W is selected from the group consisting of CH, CH₂, NH, and N;
R³ is selected from the group consisting of OH SH, NH₂, N₃, and halo;
Linker is selected from the group consisting of

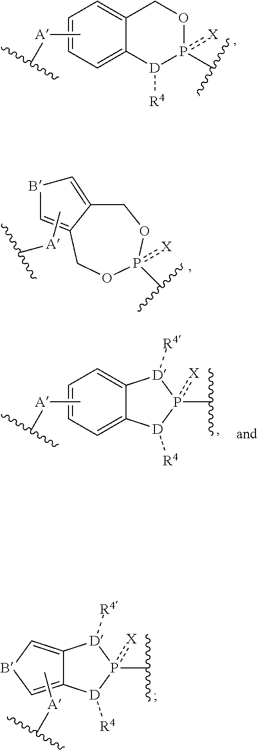

A′ is independently, at each occurrence, selected from the group consisting of
CH₂, NH, O, S, CO₃, CO₂N, CO₂S, and CO₂N;
   B′ is independently, at each occurrence, selected from the group consisting of CH₂, NH, O, and S;
   D and D′ are each independently selected from the group consisting of O, N, NH, and S;
   X is independently selected at each occurrence from the group consisting of O, NH, and S; and
R⁴ and R^{4′} are each independently selected from the group consisting of H, C₁₋₄ alkyl, C₂₋₄ alkenyl, C₂₋₄ vinyl, and C₃₋₄ allyl, all of which are optionally substituted with one, two, three, or four halo.

A nonlimiting, representative CPSI compound of Formula IV including the payload Floxuridine comprises:

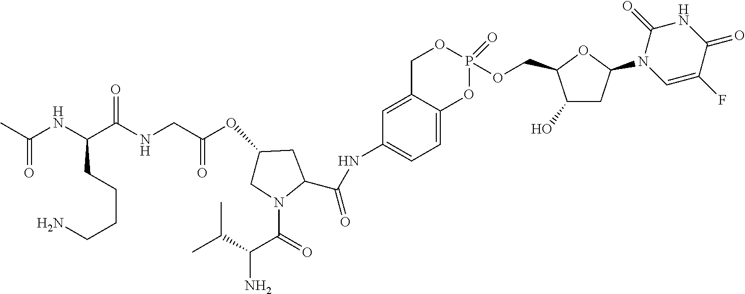 [SMILES: CC(=O)N[C@H](CCCCN)C(=O)NCC(=O)O[C@@H]1CC(C(=O)Nc2ccc3c(c2)COP(=O)(OC[C@H]2O[C@@H](n4cc(F)c(=O)[nH]c4=O)C[C@@H]2O)O3)N(C(=O)[C@H](N)C(C)C)C1]

or a pharmaceutically acceptable salt thereof.

Another nonlimiting, representative CPSI compound of Formula IV including the payload 6-thioguanine comprises:

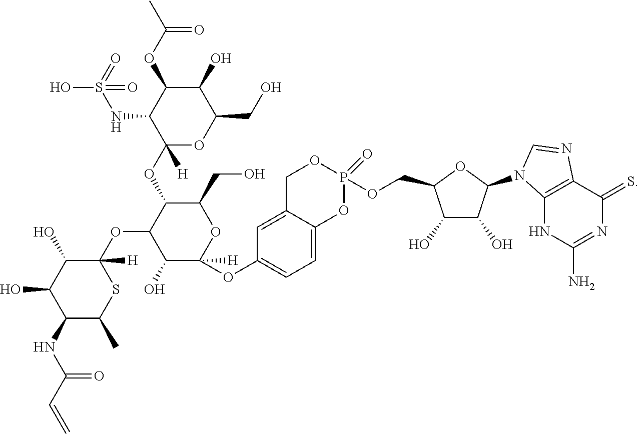 [SMILES: C=CC(=O)N[C@H]1[C@@H](O)[C@H](O)[C@H](OC2[C@@H](O)[C@H](Oc3ccc4c(c3)COP(=O)(OC[C@H]3O[C@@H](n5cnc6c(=S)nc(N)[nH]c65)[C@H](O)[C@@H]3O)O4)O[C@H](CO)[C@H]2O[C@H]2O[C@H](CO)[C@H](O)[C@H](OC(C)=O)[C@H]2NS(=O)(=O)O)S[C@H]1C]

or a pharmaceutically acceptable salt thereof.

Representative, nonlimiting metabolically activated mTOR CPSI modulators fall into the generic structures of Formula V-VIII are set forth below.

Some CPSI compounds fall into the generic structure of Formula V:

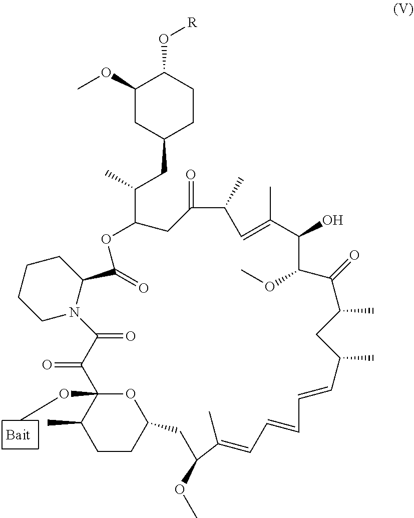

or a pharmaceutically acceptable salt thereof, wherein R is H or R is an OH substituent-containing C₁₋₈ alkyl, C₂₋₄ alkenyl, C₂₋₄ vinyl, or C₃₋₈ allyl group.

Nonlimiting, representative CPSI compounds having the structure of Formula V are selected from the group consisting of:

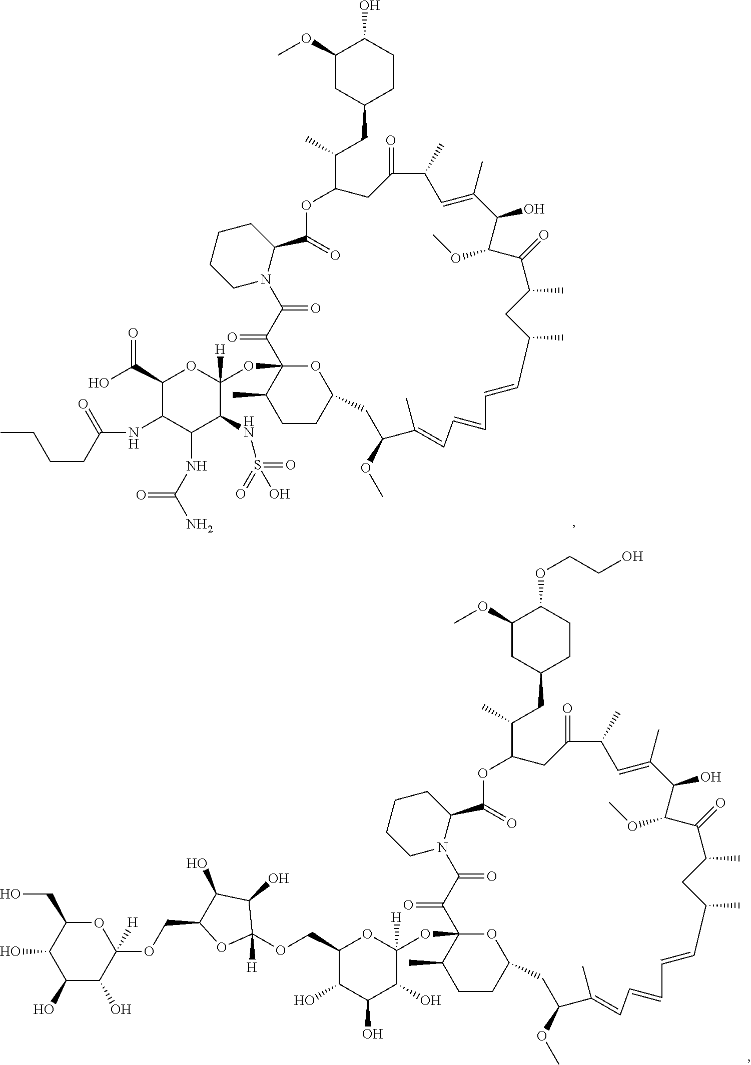

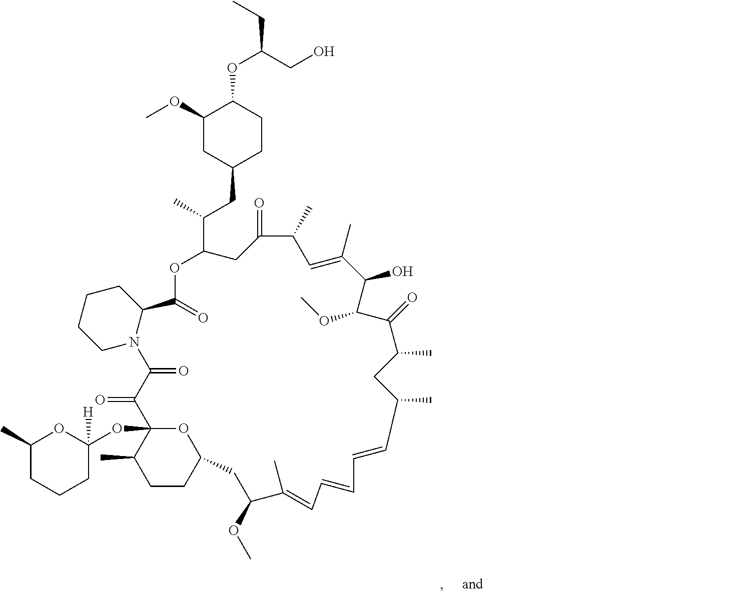

a pharmaceutically acceptable salt thereof.

Some CPSI compounds fall into the generic structure of Formula VI:

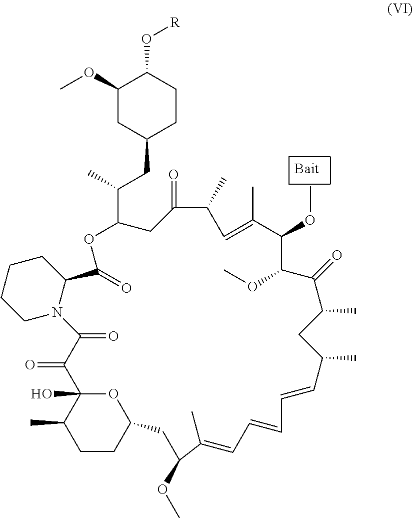

or a pharmaceutically acceptable salt thereof, wherein R is H or R is an OH substituent-containing C₁₋₈ alkyl, C₂₋₈ alkenyl, C₂₋₈ vinyl, or C₃₋₈ allyl group.

Nonlimiting, representative CPSI compounds having the structure of Formula VI are selected from the group consisting of

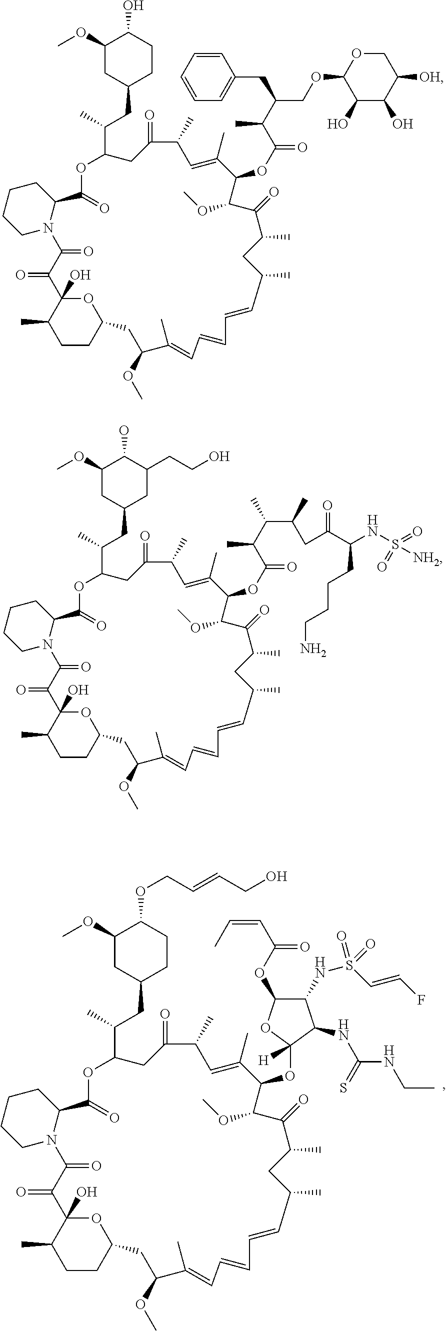

a pharmaceutically acceptable salt thereof.

Some CPSI compounds fall into the generic structure of Formula VII:

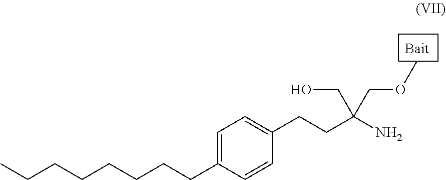 [SMILES: CCCCCCCCc1ccc(CCC(N)(CO)COC)cc1]

or a pharmaceutically acceptable salt thereof.

Nonlimiting, representative CPSI compounds having the structure of Formula VII are selected from the group consisting of

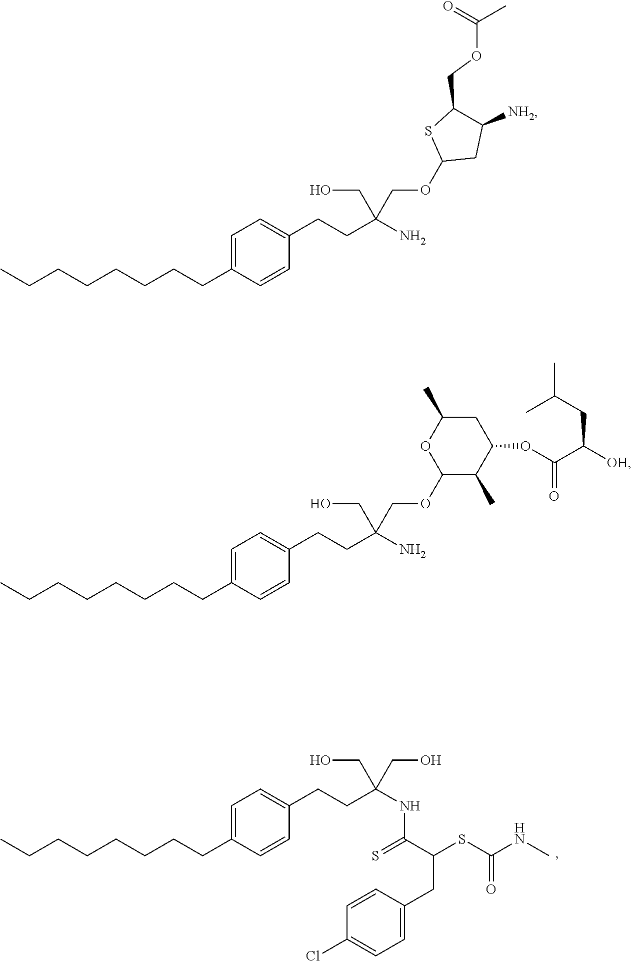 [SMILES: CCCCCCCCc1ccc(CCC(N)(CO)COC2C[C@H](N)[C@H](COC(C)=O)S2)cc1 ; CCCCCCCCc1ccc(CCC(N)(CO)COC2O[C@@H](C)C[C@H](OC(=O)[C@H](O)CC(C)C)[C@H]2C)cc1 ; CCCCCCCCc1ccc(CCC(CO)(CO)NC(=S)C(Cc2ccc(Cl)cc2)SC(=O)NC)cc1]

a pharmaceutically acceptable salt thereof.

Some CPSI compounds fall into the generic structure of Formula VIII:

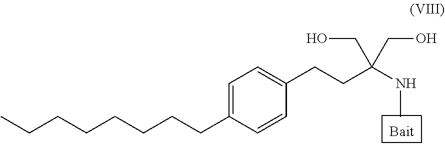 [SMILES: CCCCCCCCc1ccc(CCC(CO)(CO)NC)cc1]

or a pharmaceutically acceptable salt thereof.

Nonlimiting, representative CPSI compounds having the structure of Formula VIII are selected from the group consisting of

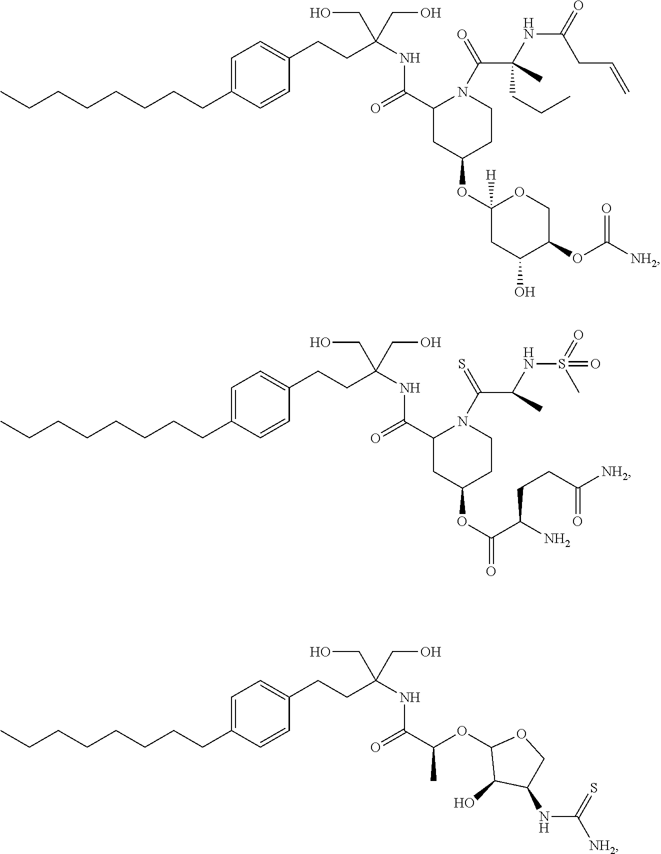 [SMILES: C=CCC(=O)N[C@](C)(CCC)C(=O)N1CC[C@@H](O[C@@H]2C[C@@H](O)[C@H](OC(N)=O)CO2)CC1C(=O)NC(CO)(CO)CCc1ccc(CCCCCCCC)cc1 ; CCCCCCCCc1ccc(CCC(CO)(CO)NC(=O)C2C[C@H](OC(=O)[C@H](N)CCC(N)=O)CCN2C(=S)[C@H](C)NS(C)(=O)=O)cc1 ; CCCCCCCCc1ccc(CCC(CO)(CO)NC(=O)[C@H](C)OC2OC[C@@H](NC(N)=S)[C@H]2O)cc1]

a pharmaceutically acceptable salt thereof.

The CPSI compounds described herein can be asymmetric (e.g., having one or more stereocenters). All stereoisomers of such compounds, such as enantiomers and diastereomers, are intended unless otherwise indicated.

CPSI compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis.

Many geometric isomers of olefins, C═N double bonds and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as +/−camphorsulfonic acid. Other resolving agents suitable for fractional crystallization methods include stereoisomerically pure forms of +/−methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

The CPSI compounds of the present disclosure can have the (R)-configuration or the (S)-configuration. In compounds with more than one chiral center, each of the chiral centers in the compound may be independently (R) or (S), unless otherwise indicated.

CPSI compounds according to the present disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

### Syntheses

Synthesis of a CPSI compound according to the disclosure includes any method known to one with skill in the art. One useful representative method is as follows.

Payloads as described herein are commercially available or are synthesized by alternations of methods known to one with skill in the art, for example, from there commercially available components via existing reported methods.

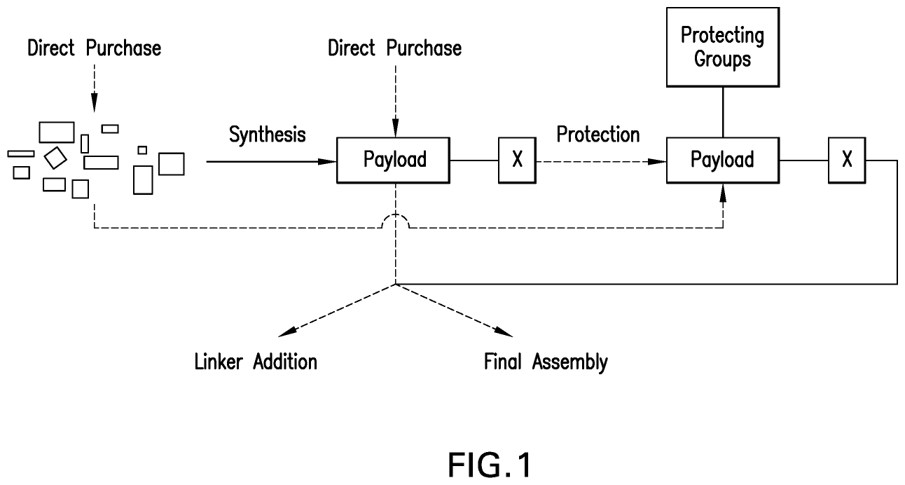

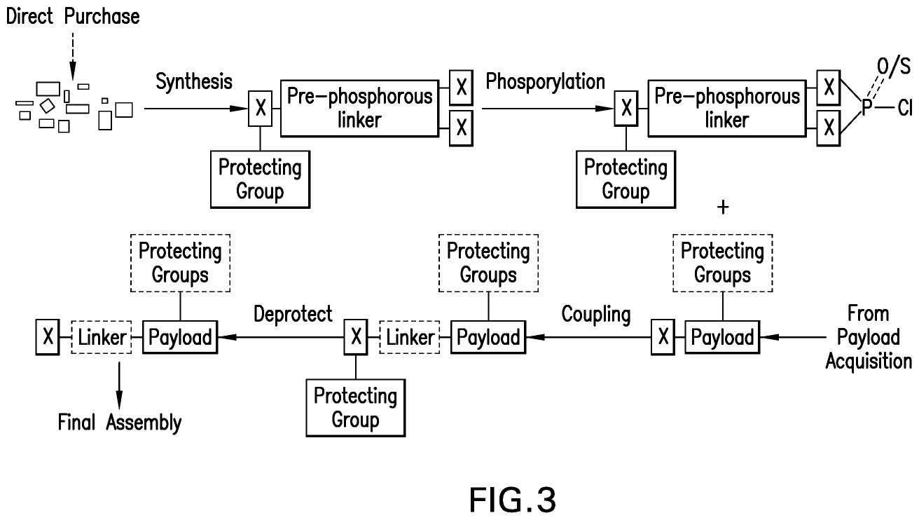

As shown in FIG. 1, payloads present a functional group (labeled “X”), and specified by the heteroatom used to attach to “Bait” in the generic structures of the CSIMs described herein. Payloads that present only one exposed functional position that is potentially active for the coupling reaction necessary to attach the Bait or the “Linker” should there be one, can be brought directly onto the Final Assembly process (FIG. 3), or the Linker Addition process (FIG. 3) as appropriate. Payloads with multiple functional groups that are potentially reactive or chemically sensitive to the Bait coupling chemistry in the Final Assembly process or the Linker Addition process, should there be one, may include other potentially active or reactive substrates that need to be protected first. This may be done directly with the completed payload via standard means (e.g., Wuts and Green; Greene's Protective Groups in Organic Synthesis, Fourth Edition; John Wiley & Sons, Inc.: 2007), or can be done directly from altering the synthesis of the payload, e.g., by omitting steps that remove protecting groups in the synthesis itself as well as adding, or by changing the protecting groups specified in the existing synthesis as necessary.

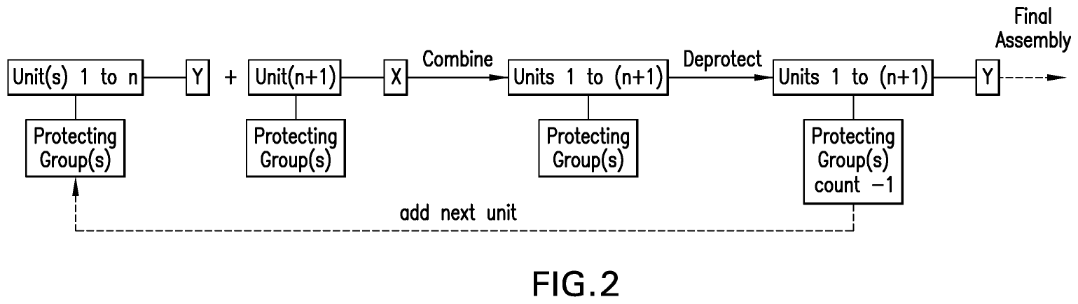

Bait components may be obtained commercially or synthesized by methods known by those with skill in the art; for Bait components that are not commercially available as ready units for use in the final assembly (e.g Fmoc-Gly-Tyr(tBu)-OH a.k.a. Pubchem ID=329767849), Baits are individually synthesized from amino acid and/or sugar components via standard methods (e.g., Jaradat (2017) Amino Acids. 50 (1); 39-68) and/or oligosaccharide synthesis (e.g., Levy et al., The Organic Chemistry of Sugar; Taylor & Francis: 2006) depending on the composition of a given bait. When Bait are built up from commercially available units, this is done in a sequential manner of coupling and deprotecting as generically outlined in FIG. 2. For a bait to be ready to be attached to either a Linker or a Payload directly, a single R5 position that is the point of attachment to the linker or payload is left unprotected (labeled as “Y” in FIG. 2), while all other aliphatic amines and protic functionality with a pKa<20 in DMSO are protected via standard means (see, e.g., Wuts and Green (2007) supra).

The optional Linker addition (FIG. 3) is carried out by synthesizing the portion of the linker missing the phosphorous found in all Linkers, the “Pre-phosphorous-linker”, synthesized by methods known to one with skill in the art from commercially available components. Pre-Phosphorous-linkers present three “X” groups, one protected that are used to couple with the Bait infera, and two unprotected that when both bound to the same phosphorous atom builds a phosphacycle from one of the bait generics supra. The Pre-phosphorous Linker is then phosphorylated with an appropriate phosphorous species (e.g., P(O)Cl₃, P(S)Cl₃, PCl₃₎, using an appropriate base (e.g., triethyl amine), and optionally a catalyst (e.g., 4-dimethylaminopyradine). This phosphorylated species is then coupled to the payload through a similar reaction using an appropriate base (e.g., triethyl amine), and optionally a catalyst (e.g., 4-dimethylaminopyradine). The protecting group on the Linker's remaining “X” group is then deprotected before the “Linker”—“Payload” partial assembly is brought onto the final assembly process.

The final assembly (FIG. 3) is carried out by coupling the appropriately protected Bait, either from the Bait Synthesis as described supra, or commercially obtained, and the Payload, with or without a linker and with or without protecting groups as necessary, described supra. This is done with an appropriate coupling reaction used for the exposed functionality on both the payload and bait (e.g., a “Bait” presenting a “Y”, a.k.a. R⁵ with a carboxyl group may be coupled to a “Payload” presenting an “X” that was an aliphatic amine with N,N′-dicyclohexylcarbodiimide (DDC)). All residual protecting groups are then removed from the resulting assembled compound to yield the active final “Bait”—“Payload” assembly.

### Disorders to be Treated

Disorders that can be treated with the CPSI compounds according to the disclosure include proliferation disorders such as, but not limited to cancers such as solid tumors and liquid cancers. Non-limiting liquid cancers that can be treated include leukemia (e.g., Acute Lymphocytic Leukemia, T-Acute Lymphocytic Leukemia, Acute Myeloid Leukemia. Hairy Cell Leukemia, Chronic Lymphocytic Leukemia, B-Chronic Lymphocytic Leukemia, Chronic Myeloid Leukemia), lymphoma (e.g., Peripheral T Cell Lymphoma, Mantel Cell Lymphoma, Non-Hodgkin's Lymphoma), Myelodysplastic Syndromes, Myeloproliferative Neoplasms, and Polycythemia vera. Representative solid cancers that can be treated with a CPSI compound according to the disclosure include, but are not limited to, Gastric Cancer, non-small cell lung cancer, Pleural Mesothelioma, Breast Cancer, Colon cancer, Pancreatic cancer, bladder cancer, Head and Neck cancer, cervical cancer, testicular cancer, Osteosarcoma, ovarian cancer, and testicular cancer.

Other proliferative disorders that can be treated are inflammatory diseases such as atherosclerosis (AS), where macrophages play a central role. Asthma (involving T cells, macrophages, neutrophils, eosinophils) can also be treated.

Particular autoimmune diseases are also treatable with these inhibitors. For example, rheumatoid arthritis (RA), inflammatory bowel diseases (IBD), celiac disease, diabetes mellitus type 1, Graves' disease, multiple sclerosis (MS), systemic lupus erythematosus (SLE), Sarcoidosis, pemphigus vulgaris (mostly B cell driven), myasthenia gravis (B cell driven), and psoriasis can be treated.

### Pharmaceutical Formulations and Treatment

The CPSIs according to the disclosure are useful in in pharmaceutical formulations that treat proliferative disorders. These pharmaceutical formulations comprise a therapeutically effective amount of a CPSI, which has anti-proliferative and immunomodulatory properties, and a pharmaceutically acceptable carrier. The formulations may also comprise two or more different CPSI compounds. For example, the formulation can comprise a CPSI compound with one payload and a different CPSI with a different payload or different payload and different bait. The formulations according to the disclosure may also comprise another cancer therapeutic different that the CPSIs of the present disclosure. Nonlimiting, exemplary therapeutic agents include TNF inhibitors, IL-6 inhibitor, IL-12 inhibitor, BLyS inhibitor CTLA4-IgGs and anti-CD20 antibodies.

The term “pharmaceutically acceptable carrier” is to be understood herein as referring to any substance that may, medically, be acceptably administered to a patient, together with ta derivative according to the disclosure, and which does not undesirably affect the pharmacological and synergistic activity of the inhibitor. A “pharmaceutically acceptable carrier” may thus be, for example, a pharmaceutically acceptable member(s) comprising of diluents, preservatives, solubilizers, emulsifiers, adjuvant, tonicity modifying agents, buffers as well as any other physiologically acceptable vehicle. These formulations are prepared with the pharmaceutically acceptable carrier in accordance with known techniques, for example, those described in Remington. The Science and Practice of Pharmacy (9th Ed. 1995).

The CPSIs of the present disclosure can also be in the form of conventional, non-toxic salts which can be prepared, for example, from non-toxic inorganic or organic acids. The pharmaceutically acceptable CSII salts of the present disclosure can be synthesized from the parent CSII which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as, but no limited to, ether, ethyl acetate, ethanol, isopropanol, or acetonitrile are useful. The phrase “pharmaceutically acceptable salt” is not limited to a mono, or 1:1, salt. For example, “pharmaceutically acceptable salt” also includes bis-salts, such as a bis-hydrochloride salt. Lists of representative suitable salts are found in Remington's Pharmaceutical Sciences, 17th ed. (Mack Publishing Company, Easton, Pa., 1985, p. 1418) and in Berge et al. (J. Pharm. Sci. (1977) 66(2):384).

For use in medicine, the salts of the anti-proliferative CPSIs are pharmaceutically acceptable salts. Other salts may, however, be useful in the preparation of the CSIIs or of their pharmaceutically acceptable salts according to the disclosure. Suitable pharmaceutical salts of the CPSIs according to the present disclosure include acid addition salts which may, for example, be formed by mixing a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Additionally, where the anti-infective CPSIs in the formulation carries an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The pharmaceutical formulations may be prepared for injectable use, topical use, oral use, intramuscular or intravenous injection, inhalation use, transdermal use, intradermal, transmembrane use, and the like. These formulations are in unit dosage forms such as tablets, pills, capsules, powders, granules, sterile parenteral solutions or suspensions, metered aerosol or liquid (nebulized) sprays, drops, ampoules, auto-injector devices or suppositories; for oral parenteral, intranasal, sublingual topical or rectal administration, or for administration by inhalation or insufflation. Alternatively, the formulations may be presented in a form suitable for one-weekly or once-monthly administration; for example, an insoluble salt of the derivative, such as decanoate salt, may be adapted to provide a depot preparation for intramuscular injection. An erodible polymer containing the inhibitor may also be envisaged.

For preparing solid compositions such as tablets, the CPSI is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a CSII of the present disclosure, or a pharmaceutically acceptable salt thereof.

These formulations may be homogeneous, i.e., the derivative is dispersed evenly throughout the composition so that the composition may be readily subdivided into equally effective unit dosage forms such as tablets, pills and capsules. This solid formulation composition is then subdivided into unit dosage forms of the type described above containing from about 0.1 mg to about 500 mg of the CPSI. Some useful unit dosage forms contain from about 1 mg to about 100 mg, for example, about 1 mg, about 2 mg, about 5 mg, about 10 mg, about 25 mg, about 50 mg, or about 100 mg, of the CPSI. The tablets or pills comprising the CPSIs can be coated or otherwise compounded to provide a dosage form affording the advantage of prolonged action. The liquid forms in which the CPSIs of the present disclosure may be incorporated for administration orally or by injection include aqueous solutions, suitably flavored syrups, aqueous or oil suspensions, and flavored emulsions with edible oils as well as elixirs and similar pharmaceutical vehicles.

Injectable dosage forms may be sterilized in a pharmaceutically acceptable fashion, for example by steam sterilization of an aqueous solution sealed in a vial under an inert gas atmosphere at 120° C. for about 15 minutes to about 20 minutes, or by sterile filtration of a solution through an about 0.2 μM or smaller pore-size filter, optionally followed by a lyophilization step, or by irradiation of a composition containing an inhibitor of the present disclosure by means of emissions from a radionuclide source.

A therapeutically effective dosage of the formulation according to the disclosure depends on the disorder treated and may vary from patient to patient, and factors such as the age and physical size of the patient, the patient's genetics, and the diagnosed condition of the patient, and the route of delivery of the dosage form to the patient. A therapeutically effective dose and frequency of administration of a dosage form may be determined in accordance with routine pharmacological procedures known to those skilled in the art. For example, dosage amounts and frequency of administration may vary or change as a function of time and severity of the disorder. A dosage from about 0.1 mg/kg to about 1000 mg/kg, or from about 1 mg/kg to about 100 mg/kg inhibitor may be suitable. For example, for the treatment of proliferation disorders, a suitable dosage level is about 0.001 mg/kg to about 250 mg/kg per day. The formulation maybe administered as a bolus and/or a regimen of about 1 to about 4 times per day.

Alternatively, when treating a subject with a CPSI formulation, treatment may also include administering another formulation comprising another CPSI and/or an anti-proliferative compound, that is different than the CPSI in the formulation as set forth above.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A cancer- or proliferative cell-selective inhibitor or modulator compound (“CPSI”) comprising:
a bait comprising a molecule which targets a cancer cell or a cell affected by a proliferative disorder, the bait comprising a substrate cleavable by a disease factor present in the cancer cell or the cell affected by a proliferative disorder; and
a payload comprising an active inhibitor or modulator of a metabolic enzyme found in the cancer cell or in the cell affected by a proliferation disorder, the payload comprising an antiproliferative agent.

2. The CPSI compound of claim 1, wherein the bait is selected from an oligomer comprising 1-5 units, which are, independently at each occurrence selected from the group consisting of 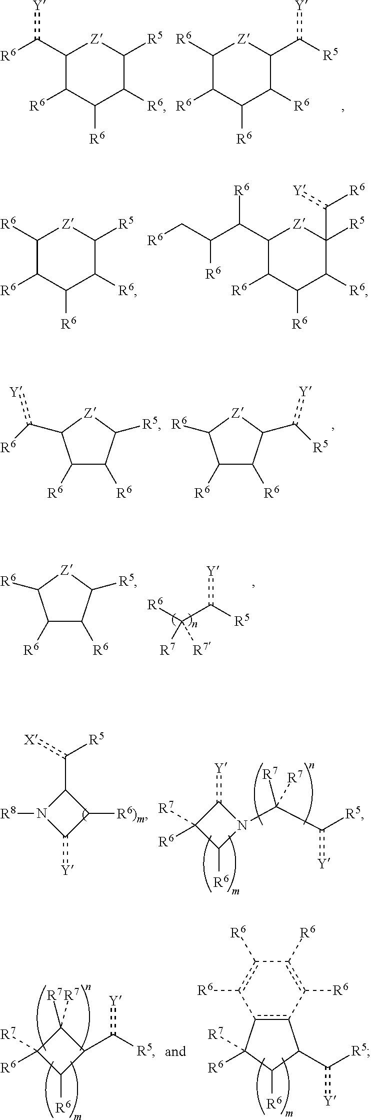
wherein:
Z′ is independently, at each occurrence, selected from the group consisting of CH₂, NH, O, and S;
X′ and Y′ are independently, at each occurrence, selected from the group consisting of O, NH, and S;
R⁵ is independently, at each occurrence, selected from the group consisting of a bond to any R⁶, a bond to any R⁸, and an attachment to any payload;
R⁶ is independently, at each occurrence, selected from the group consisting of H, —R^{D}, —R^{A}—R⁵, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{D} is independently, at each occurrence, selected from the group consisting of OH, SH, NH₂, N₃, and halo;
R⁷ and R⁷′ are independently, at each occurrence, selected from the group consisting of H, NH₂, OH, C₆₋₁₀ aryl, 5-10 membered heteroaryl, C₁₋₄ alkyl, —(CH₂)₁₋₃—C₆₋₁₀ aryl, and —(CH₂)₁₋₃-5-10 membered heteroaryl; wherein aryl, heteroaryl, and alkyl are optionally substituted with one, two, or three substituents selected from the group consisting of halo, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, —R^{A}—(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵;
m is 0, 1, 2, 3, or 4; and
n is 0, 1, 2, or 3.

3. The CPSI compound of claim 2, wherein the Bait is selected from the group consisting of:

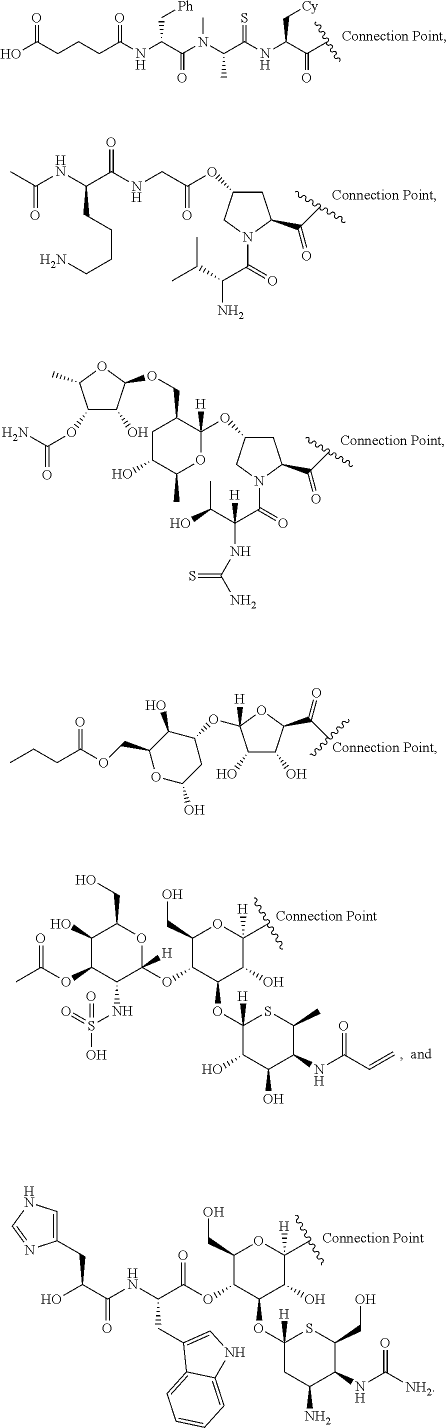

4. The CPSI compound of claim 1, wherein the payload or as metabolically active species thereof, modulates or inhibits an enzyme selected from the group consisting of thymidylate synthase, proteasome, dihydrofolate reductase, ribonucleotide reductase, DNA methyltransferase, glycinamide ribonucleotide formyltransferase, adenosine deaminase, glutamine-phosphoribosyl pyrophosphate, amidotransferase, tyrosine-protein kinase, spleen tyrosine kinase, phosphatidylinositol 3-kinases, phosphoinositide 3-kinases, Bruton's tyrosine kinase, histone deacetylases, Janus kinase, XPO1, and bromodomain and extra-terminal domain (BET) proteins.

5. The CPSI compound of claim 1, wherein the payload modulates or inhibits a receptor selected from the group consisting of epidermal growth factor receptor, mammalian target of rapamycin (mTOR), and BCL2.

6. The CPSI compound of claim 1, wherein the payload comprises Bortezomib or a derivative thereof.

7. The CPSI compound claim 6, which comprises: 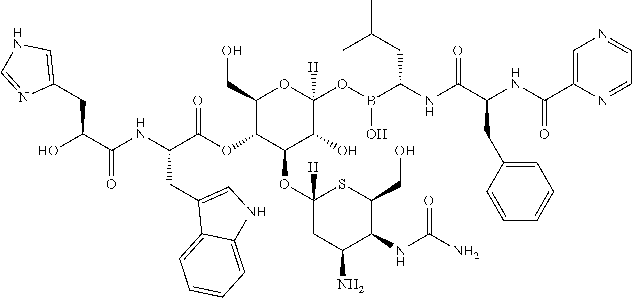 [SMILES: CC(C)C[C@H](NC(=O)[C@H](Cc1ccccc1)NC(=O)c1cnccn1)B(O)O[C@@H]1O[C@H](CO)[C@@H](OC(=O)[C@H](Cc2c[nH]c3ccccc23)NC(=O)[C@@H](O)Cc2c[nH]cn2)[C@H](O[C@H]2C[C@H](N)[C@H](NC(N)=O)[C@H](CO)S2)[C@H]1O]
or a pharmaceutically acceptable salt thereof.

8. The CPSI compound of claim 1, wherein the payload comprises Lestaurtinib or a derivative thereof.

9. The CPSI compound claim 7, which comprises: 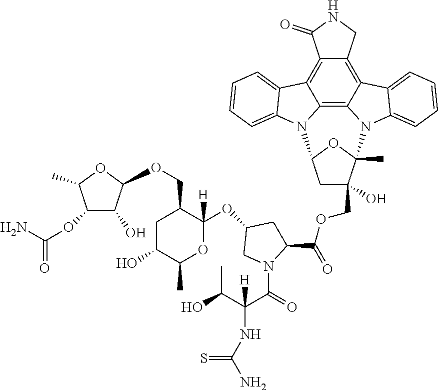 [SMILES: C[C@H](O)[C@@H](NC(N)=S)C(=O)N1C[C@H](O[C@@H]2O[C@@H](C)[C@H](O)C[C@H]2CO[C@@H]2O[C@@H](C)[C@@H](OC(N)=O)[C@H]2O)C[C@H]1C(=O)OC[C@@]1(O)C[C@H]2O[C@]1(C)n1c3ccccc3c3c4c(c5c6ccccc6n2c5c31)C(=O)NC4]
or a pharmaceutically acceptable salt thereof.

10. The CPSI compound of claim 1, wherein the payload comprises Umbralisib or a derivative thereof.

11. The CPSI compound of claim 10, which comprises: 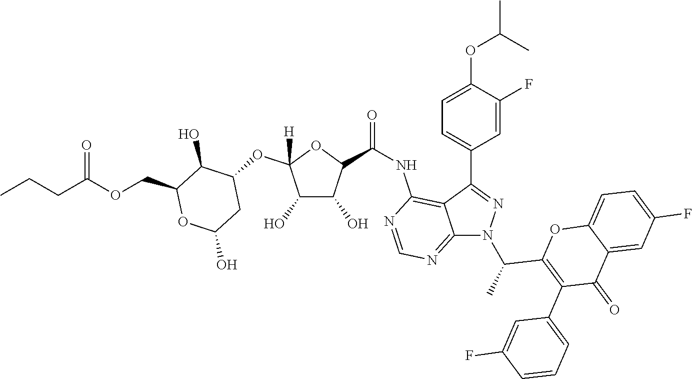
or a pharmaceutically acceptable salt thereof.

12. The CPSI compound of claim 1, wherein the payload comprises Floxuridine or a derivative thereof.

13. The CPSI compound of claim 12, which comprises: 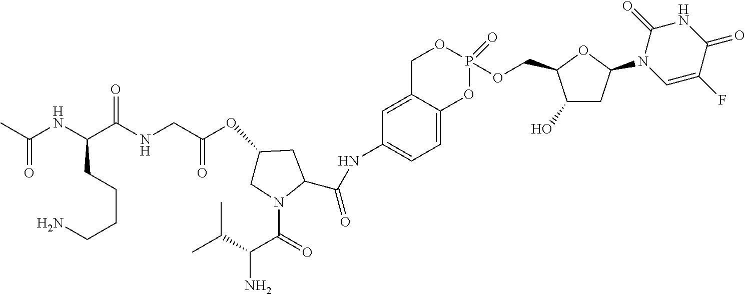 [SMILES: CC(=O)N[C@H](CCCCN)C(=O)NCC(=O)O[C@@H]1CC(C(=O)Nc2ccc3c(c2)COP(=O)(OC[C@H]2O[C@@H](n4cc(F)c(=O)[nH]c4=O)C[C@@H]2O)O3)N(C(=O)[C@H](N)C(C)C)C1]
or a pharmaceutically acceptable salt thereof.

14. The CPSI compound of claim 1, wherein the payload comprises 6-thioguanine or a derivative thereof.

15. The CPSI compound of claim 14, which comprises: 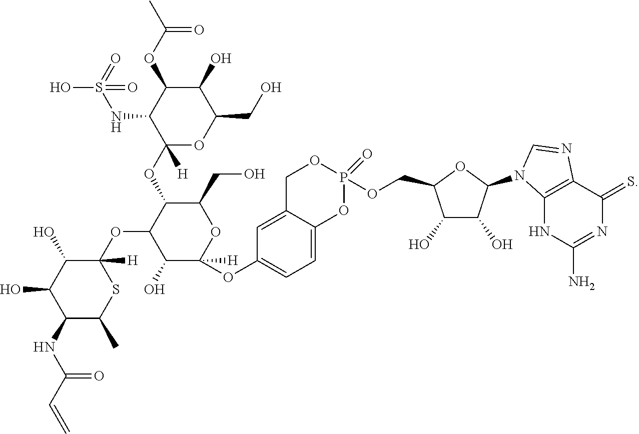 [SMILES: C=CC(=O)N[C@H]1[C@@H](O)[C@H](O)[C@H](OC2[C@@H](O)[C@H](Oc3ccc4c(c3)COP(=O)(OC[C@H]3O[C@@H](n5cnc6c(=S)nc(N)[nH]c65)[C@H](O)[C@@H]3O)O4)O[C@H](CO)[C@H]2O[C@H]2O[C@H](CO)[C@H](O)[C@H](OC(C)=O)[C@H]2NS(=O)(=O)O)S[C@H]1C]
or a pharmaceutically acceptable salt thereof.

16. The CPSI compound of claim 1, further comprising a linker molecule attached to the bait and attaching the payload to the bait.

17. The CPSI compound of claim 16, wherein the linker is selected from the group consisting of 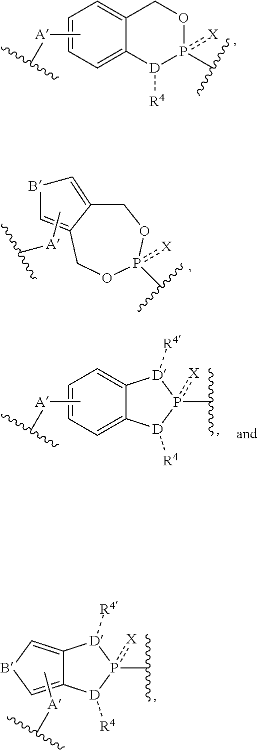
wherein:
A′ and B′ are each independently selected from the group consisting of CH₂, NH, O, and S;
D and D′ are each independently selected from the group consisting of O, N, NH, and S;
X is selected from the group consisting of O, NH, and S; and
R⁴ and R⁴′ are each independently selected from the group consisting of H, C₁₋₄ alkyl, C₂₋₄ alkenyl, C₁₋₄ vinyl, and C₁₋₄ allyl, all of which are optionally substituted with one, two, three, or four halo.

18. A pharmaceutical formulation comprising a CPSI compound of any one of claims 1-17, or a pharmaceutically acceptable salt thereof, and a pharmaceutically acceptable carrier.

19. The pharmaceutical formulation of claim 18, further comprising an anti-proliferative compound that is not the CPSI compound.

20. A method of treating a cancer or proliferative disorder in a patient, comprising administering to the patient an amount of the pharmaceutical formulation of claim 18 or 19 effective to reduce or inhibit at least one symptom of the cancer or proliferative disorder.

21. A method of treating a cancer or proliferative disorder in a patient, comprising administering to the patient a compound of any one of claims 1-17 in a pharmaceutically acceptable carrier in an amount effective to inhibit or reduce at least one symptom of the cancer or proliferative disorder.

22. The method of claim 20 or 21, wherein the cancer is a solid cancer or a liquid cancer.

23. The method of claim 22, wherein the liquid cancer is Acute Lymphocytic Leukemia, T-Acute Lymphocytic Leukemia, Peripheral T Cell Lymphoma, Acute Myeloid Leukemia, Myelodysplastic Syndromes, Hairy Cell Leukemia, Mantel Cell Lymphoma, Chronic Lymphocytic Leukemia, B-Chronic Lymphocytic Leukemia, Chronic Myeloid Leukemia, Non-Hodgkin's Lymphoma, Myeloproliferative Neoplasms, or Polycythemia vera.

24. The method of claim 22, wherein the cancer is gastric cancer, Non-Small Cell Lung Cancer, Pleural Mesothelioma, breast cancer, colon cancer, pancreatic cancer, bladder cancer, cervical cancer, Osteosarcoma, Head and Neck cancer, testicular cancer, prostate cancer or ovarian cancer.

25. The method of claim 20 or 21, further comprising administering to the patient a therapeutically effective amount of an anti-proliferative agent that is not a CPSI.

26. A method of synthesizing a CPSI compound of claim 1, comprising carrying out the method set forth in FIGS. 1-3.

27. A method of modulating the activity of a cancer or proliferative cell, comprising contacting the cell with a CPSI compound of any ne of claim 1-17, the compound reducing or inhibiting the proliferative activity of the cell.
